(19) United States
(12) Patent Application Publication
(10) Pub. No.: US 2017/0165287 A1
(43) Pub. Date: Jun. 15, 2017

(54) CRYSTAL FORM OF A NUCLEOSIDE INHIBITOR OF HCV

(71) Applicant: JANSSEN SCIENCES IRELAND UC, Co Cork (IE)

(72) Inventors: Jennifer ALBANEZE-WALKER, Mol (BE); Xianglu ZENG, Shanghai (CN); Yuanyuan YUAN, Shanghai (CN)

(21) Appl. No.: 15/039,388

(22) PCT Filed: Nov. 28, 2013
(86) PCT No.: PCT/CN2013/088077

§ 371 Date: May 25, 2016

(51) Int. Cl. A61K 31/7072; C07H 19/11

(52) U.S. Cl. CPC A61K 31/7072; C07H 19/11

(57) ABSTRACT

Provided are crystalline forms of the compound of formula (I), which is a nucleoside inhibitor of HCV, processes for the preparation thereof, and pharmaceutical compositions comprising these crystalline forms.

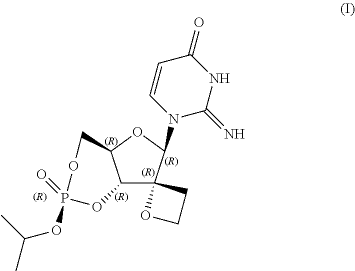

### FIELD OF THE INVENTION

The present invention relates to crystal forms of a nucleoside inhibitor of HCV.

### BACKGROUND OF THE INVENTION

HCV is a single stranded, positive-sense RNA virus belonging to the Flaviviridae family of viruses in the hepacivirus genus. The NS5B region of the RNA polygene encodes a RNA dependent RNA polymerase (RdRp), which is essential to viral replication. Following the initial acute infection, a majority of infected individuals develop chronic hepatitis because HCV replicates preferentially in hepatocytes but is not directly cytopathic. In particular, the lack of a vigorous T-lymphocyte response and the high propensity of the virus to mutate appear to promote a high rate of chronic infection. Chronic hepatitis can progress to liver fibrosis, leading to cirrhosis, end-stage liver disease, and HCC (hepatocellular carcinoma), making it the leading cause of liver transplantations. There are six major HCV genotypes and more than 50 subtypes, which are differently distributed geographically. HCV genotype 1 is the predominant genotype in Europe and in the US. The extensive genetic heterogeneity of HCV has important diagnostic and clinical implications, perhaps explaining difficulties in vaccine development and the lack of response to current therapy.

Transmission of HCV can occur through contact with contaminated blood or blood products, for example following blood transfusion or intravenous drug use. The introduction of diagnostic tests used in blood screening has led to a downward trend in post-transfusion HCV incidence. However, given the slow progression to the end-stage liver disease, the existing infections will continue to present a serious medical and economic burden for decades.

Therapy possibilities have extended towards the combination of a HCV protease inhibitor (e.g. Telaprevir or boceprevir) and (pegylated) interferon-alpha (IFN-α)/ribavirin. This combination therapy has significant side effects and is poorly tolerated in many patients. Major side effects include influenza-like symptoms, hematologic abnormalities, and neuropsychiatric symptoms. Hence there is a need for more effective, convenient and better-tolerated treatments.

The NS5B RdRp is essential for replication of the single-stranded, positive sense, HCV RNA genome. This enzyme has elicited significant interest among medicinal chemists. Both nucleoside and non-nucleoside inhibitors of NS5B are known. Nucleoside inhibitors can act as a chain terminator or as a competitive inhibitor, or as both. In order to be active, nucleoside inhibitors have to be taken up by the cell and converted in vivo to a triphosphate. This conversion to the triphosphate is commonly mediated by cellular kinases, which imparts additional structural requirements on a potential nucleoside polymerase inhibitor. In addition this limits the direct evaluation of nucleosides as inhibitors of HCV replication to cell-based assays capable of in situ phosphorylation.

Several attempts have been made to develop nucleosides as inhibitors of HCV RdRp, but while a handful of compounds have progressed into clinical development, none have proceeded to registration. Amongst the problems which HCV-targeted nucleosides have encountered to date are toxicity, mutagenicity, lack of selectivity, poor efficacy, poor bioavailability, sub-optimal dosage regimes and ensuing high pill burden and cost of goods.

Spirooxetane nucleosides, in particular 1-(8-hydroxy-7-(hydroxy-methyl)-1,6-dioxaspiro[3.4]octan-5-yl)pyrimidine-2,4-dione derivatives and their use as HCV inhibitors are known from WO2010/130726, and WO2012/062869, including CAS-1375074-52-4.

There is a need for HCV inhibitors that may overcome at least one of the disadvantages of current HCV therapy such as side effects, limited efficacy, the emerging of resistance, and compliance failures, or improve the sustained viral response.

The present invention concerns HCV-inhibiting uracyl spirooxetane derivatives with useful properties regarding one or more of the following parameters: antiviral efficacy towards at least one of the following genotypes 1a, 1b, 2a, 2b, 3, 4 and 6, favorable profile of resistance development, lack of toxicity and genotoxicity, favorable pharmacokinetics and pharmacodynamics and ease of formulation and administration.

Such an HCV-inhibiting uracyl spirooxetane derivative is a compound with formula I

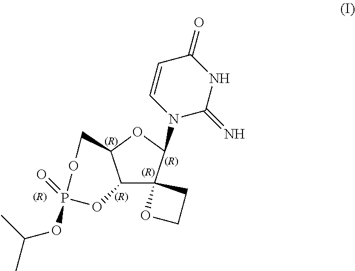

including any pharmaceutically acceptable salt or solvate thereof.

It now has been found that the compound of formula (I) can be converted into a crystalline form, which can advantageously be used as active ingredients in anti-HCV therapy.

An amorphous form is a form in which a three-dimensional long-range order does not exist. In the amorphous form the position of the molecules relative to one another are essentially random, i.e. without regular arrangement of the molecules in a lattice structure. Amorphous materials may have interesting properties, but generating and stabilizing this state usually offers difficulties in that the crystalline state typically is the more stable state. Compounds in amorphous form can convert partially or completely to crystalline forms over time or under the influence of external factors such as temperature, humidity, traces of crystalline material in the environment, etc. Usually a crystalline form of an active ingredient is preferred in the manufacture and storage of pharmaceutical dosage forms.

A crystal or crystalline form is the form in which the position of the molecules relative to one another is organized according to a three-dimensional lattice structure. Crystalline forms may include polymorphs and pseudopolymorphs. Polymorphs are different crystalline forms of the same compound resulting from a different arrangement of the molecules in the solid state.

Solid state chemistry is of interest to the pharmaceutical industry, in particular as concerns the development of suitable dosage forms. Solid state transformations may seriously impact the stability of pharmaceuticals (shelf-life). A metastable pharmaceutical solid form can change into a crystalline structure (e.g. from amorphous to crystalline) or solvate/desolvate in response to changes in environmental conditions, processing, or over time.

During the clinical development of pharmaceutical drugs, if the solid form is not held constant, the exact dosage form used or studied may not be comparable from one lot to another. It is also desirable to have processes for producing a compound with the selected solid form in high purity when the compound is used in clinical studies or commercial products since impurities present may produce undesired toxicological effects. Certain forms may exhibit enhanced thermodynamic stability or may be more readily manufactured in high purity in large quantities, and thus are more suitable for inclusion in pharmaceutical formulations.

It is an object of the present invention to provide the HCV inhibitory agent of formula (I) in a crystalline form having beneficial properties in terms of one or more of the following: safety, the ability to be formulated, to be stored and to be administered as to effectively excert its antiviral properties.

### DESCRIPTION OF THE FIGURES

FIG. 1 is an X-ray powder Diffraction (XPRD) pattern representation of Form A.

FIG. 2 is an X-ray powder Diffraction (XPRD) pattern representation of Form A and the amorphous form.

FIG. 3 is a Differential Scanning calorimetry (DSC) curve of Form A.

FIG. 4 is a thermogravimetric analysis (TGA) curve of Form A.

FIG. 5 is an IR spectrum representation of Form A.

### DESCRIPTION OF THE INVENTION

The present invention relates to an HCV inhibitor, which is the compound of formula (I) in crystalline form. The formation of such a crystalline form proved to be challenging and a suitable form did not emerge from conventional high throughput crystallization screening.

The invention in particular concerns the crystalline form of the compound of formula (I) that is denominated as Form A, or in short “Form A”.

Form A has an X-ray powder diffraction pattern comprising peaks at 11.4°±0.3°, 13.1°±0.3°, 14.8°±0.3°, 15.9°±0.3°, 16.5°±0.3°, 18.8°±0.3°, 19.8°±0.3°, 20.7°±0.3° and 21.6°±0.3° two theta.

The present invention relates as well to mixtures a crystalline form of the compound of formula (I) and the amorphous form of the compound of formula (I).

The present invention further relates to processes for preparing the crystalline form of the compound of formula (I).

The present invention also relates to a crystalline form of the compound of formula (I) for use as a medicament. This invention also relates to a crystalline form of the compound of formula (I) for use as a HCV inhibitor, or for use in the treatment of HCV-related conditions. The invention also relates to the use of a crystalline form of the compound of formula (I) in the manufacture of a medicament for inhibiting HCV, or for the treatment of HCV-related conditions. The invention furthermore provides a method of treating a mammal suffering from HCV-related conditions comprising administering an effective amount of the crystalline forms of the compound of formula (I), mixtures thereof, to said mammal. The mammal preferably is a human.

Furthermore, the invention provides a pharmaceutical composition comprising a crystalline form of the compound of formula (I), or in particular a form selected from Form A, and a pharmaceutically acceptable carrier. The said crystalline form of the compound of formula (I) preferably is present in an effective amount, i.e. an amount that is effective in preventing or treating HCV infection or conditions associated with HCV infection.

Detailed description of Form A

As indicated, the invention in particular concerns the crystalline form of the compound of formula (I) that is denominated as Form A, or in short “Form A”.

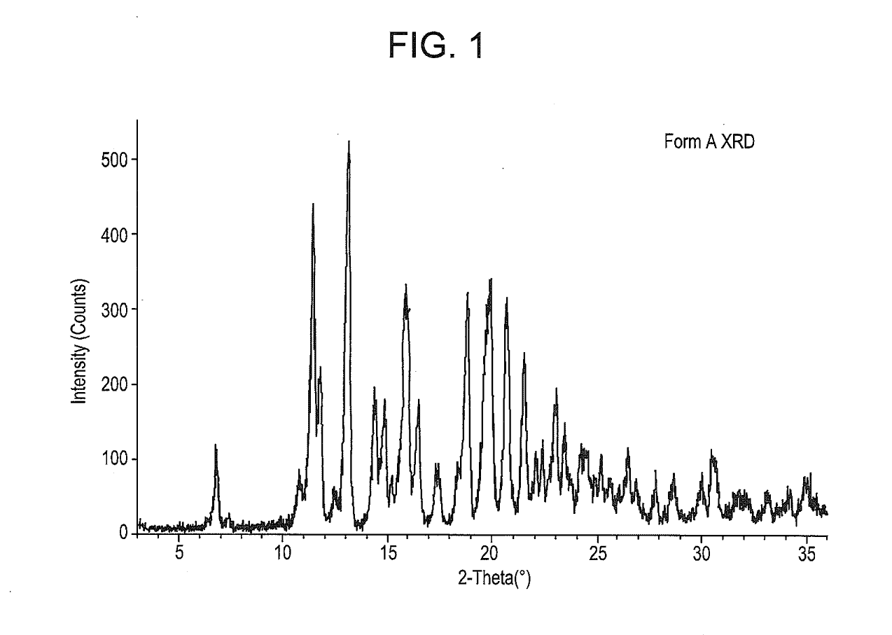

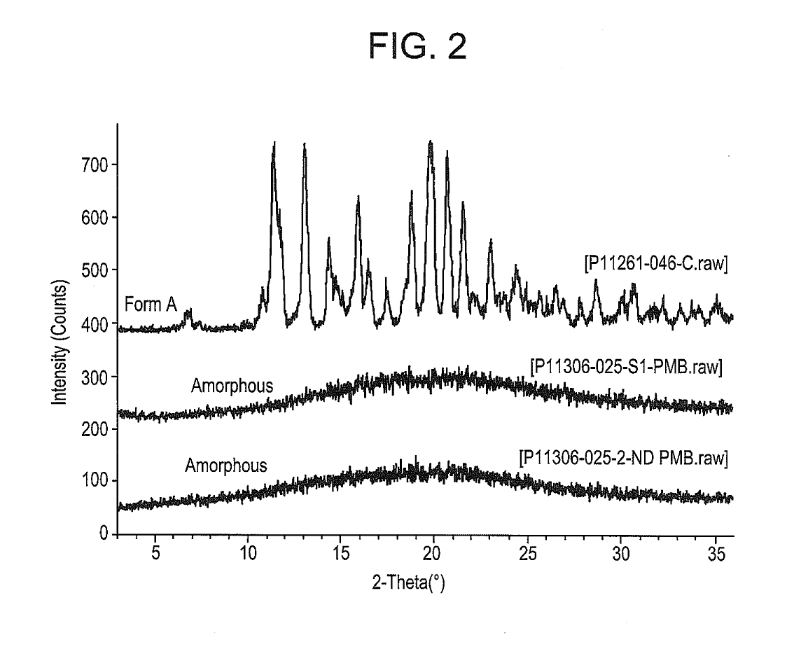

Form A has an X-ray powder diffraction pattern comprising peaks at 11.4°±0.3°, 13.1°±0.3°, 14.8°±0.3°, 15.9°±0.3°, 16.5°±0.3°, 18.8°±0.3°, 19.8°±0.3°, 20.7°±0.3° and 21.6°±0.3° two theta. The X-ray powder diffraction pattern of Form A is as substantially depicted in FIG. 1. A direct comparison with 2 different batches of amorphous compound of Formula (I) is depicted in FIG. 2. The complete overview of characterizing XPRD intensity peak positions (in degrees 2-theta) of the crystal form A are shown in the following table 1.

TABLE 1
XPRD intensity peak positions of form A

| 2-Theta (°) | Intensity (%) |
|---|---|
| 6.7 | 34 |
| 6.9 | 44 |
| 10.8 | 45 |
| 11.4 | 274 |
| 11.7 | 153 |
| 13.1 | 262 |
| 14.4 | 128 |
| 14.8 | 173 |
| 15.2 | 15 |
| 15.9 | 473 |
| 16.5 | 171 |
| 17.4 | 52 |
| 18.8 | 291 |
| 19.8 | 211 |
| 20.7 | 233 |
| 21.6 | 153 |
| 22.1 | 23 |
| 23.1 | 106 |
| 23.5 | 43 |
| 24.2 | 73 |
| 24.5 | 62 |
| 24.9 | 30 |
| 25.6 | 19 |
| 26.5 | 46 |
| 26.9 | 30 |
| 27.8 | 25 |
| 28.6 | 43 |
| 30.0 | 26 |
| 30.5 | 84 |
| 33.2 | 24 |
| 34.2 | 25 |
| 34.8 | 26 |
| 35.1 | 69 |

The X-ray powder Diffraction pattern (FIG. 1) was obtained on a Rigaku Miniflex Diffraction System (Rigaku MSC Inc.). The powder samples were deposited on a zero-background polished sample holder. A normal focus copper x-ray tube at 0.45 kW equipped with a Ni K filter scanning at 5 degrees/minute from 3.00 to 36.00 degree 2-theta was used as the x-ray source. The data processing was done using Jade 6.0 software. The relative intensities of the XRD peaks can vary depending on the sample preparation technique, crystal size distribution, various filters used, the sample mounting procedure, and the particular instrument employed. Also some new peaks may be seen or some peaks may disappear depending on the type of the machine and settings (including filters). It is commonly accepted that XTD peaks may shift to either side with 2-theta±0.3° degrees. Therefore, the accuracy of the XPRD peak positions provided for Form A is defined as ±0.3° due to experimental differences, such as instrumentations, sample preparations, and the like.

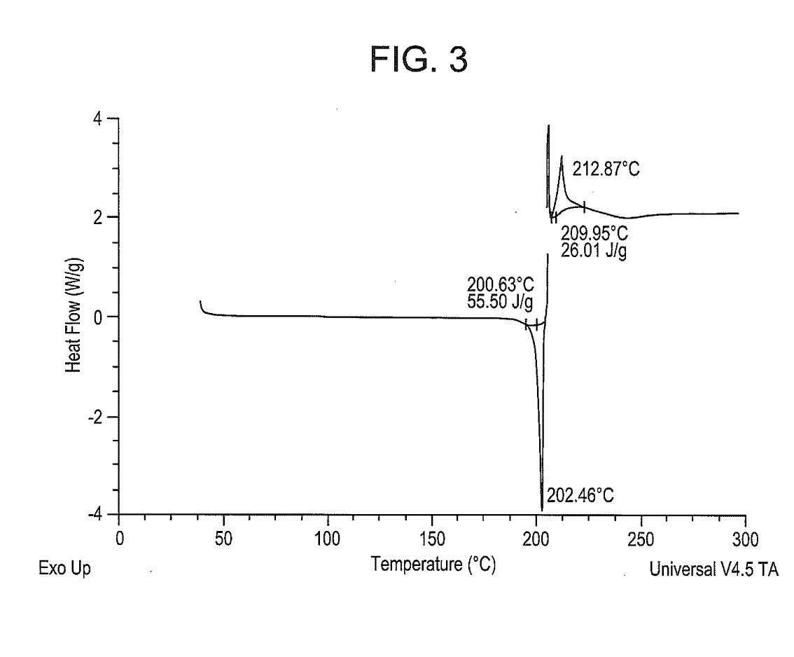

The DSC curve of Form A was determined and is as substantially depicted in FIG. 3. The characterizing DSC endothermic melt positions (in ° C.) of Form A are shown in the following table 2.

TABLE 2
DSC endothermic melt positions or ranges of the
polymorph forms of the compound of formula (I)

| | Form A |
|---|---|
| DSC endothermic melt onset (in ° C.) | 200.6 |
| DSC endothermic peak (in ° C.) | 202.5 |

DSC data were collected using a Q2000 DSC (TA instruments). Typically 3 mg of sample was used in a semi-hermetically sealed aluminum pan (no pin-hole). The sample was heated from 40° C. to 300° C. at a ramp rate of 10° C./min. The location of DSC peak may be slightly shifted depending on the particle size distribution, type of machine, and the heating rate. The presence of impurities may also change the peak location.

The tolerance of the DSC curves provided for Form A is defined as 3° C. due to experimental differences, such as instrumentation, sample preparation, and the like.

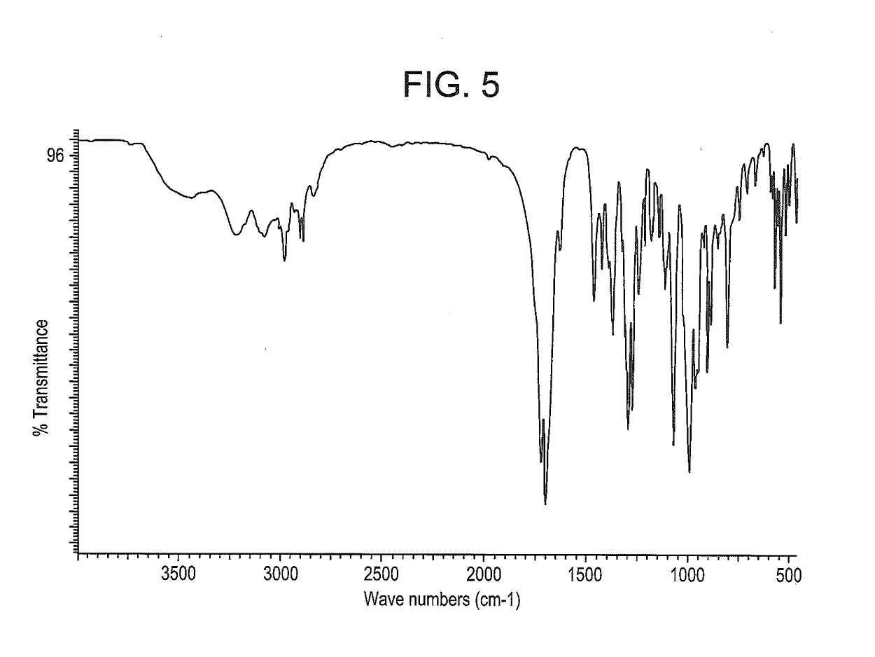

FIG. 5 shows an IR spectrum representation of Form A. The Fourier Transform Infrared (FT-IR) analysis was performed on a Thermo Nexus 670 FT-IR spectrometer. The spectrum was recorded at a resolution of 1 cm⁻¹ from a KBr pellet and represents the sum of 32 scans, background subtracted

In one embodiment, the invention provides the crystal forms designated Form A of the compound of formula (I), as specified above, substantially free from impurities. In a particular embodiment, these forms contain no more than 10% of impurities, or no more than 5% of impurities, or no more than 1% of impurities, or no more than 0.5% of impurities, or no more than 0.1% of impurities. The impurities may be other compounds or may the amorphous form.

This invention further provides a mixture of a crystalline form of the compound of formula (I) and the amorphous form of the compound of formula (I). In one embodiment, there is provided a mixture comprising Form A and the amorphous form of the compound of formula (I).

### DETAILED DESCRIPTION OF THE PROCESS ACCORDING TO THE INVENTION

The crystal form of a compound with formula (I) can be prepared in multiple ways.

In one embodiment, the process for preparing a crystal form of a compound with formula (I) comprises:
a) Dissolving compound of formula (I) in a solvent;
b) Subsequently adding an anti-solvent until crystal formation is observed.

A solvent is defined as a solvent in which the compound of formula (I) has a high solubility at the used temperature. Preferably, high solubility indicates solubility over 200 mg/ml of solvent, even more preferably over 200 mg/ml of solvent at room temperature.

In a particular embodiment, the solvent is selected from the group comprising Dichloromethane, Tetrahydrofuran or Ethanol or a mixture thereof.

An anti-solvent is defined as a solvent in which the compound of formula 1 has a low solubility at the used temperature. Preferably, low solubility indicates a solubility lower than 200 mg/ml of solvent, even more preferably lower than 150 mg/ml of solvent at room temperature.

In a particular embodiment, the anti-solvent is selected from the group comprising Ethyl acetate, n-Heptane, Isopropyl Acetate, Methyl tert-butyl ether or a mixture thereof.

In a further embodiment, the compound of formula (I) is dissolved in least 5 volumes Dichloromethane, and at least 20 volumes of Ethyl acetate, n-Heptane or Ethyl acetate/n-Heptane mixture are subsequently added.

In another embodiment, the process for preparing a crystal form of a compound with formula (I) comprises:
a) Preparing a suspension or slurry of the amorphous form of the compound of formula (I) in Ethyl acetate, n-Heptane or an Ethyl acetate/n-Heptane mixture;
b) Stirring the suspension or slurry for at least one hour.

In addition, such suspension or slurry can be seeded with crystal seeds, preferably seeds of Form A.

The invention provides as well a process wherein the obtained crystalline form is isolated by filtration or centrifugation, optionally combined with washing and drying.

The starting material used for the processes of the present invention may be a crystalline or amorphous form of the compound of formula (I). With crystallization processes, the crystalline form of the starting material does not usually affect the final result. With trituration, the final product may vary depending on the starting material. The one of skill in the art would appreciate the convenient manipulation of the starting material to obtain a desirable form with trituration. The present invention is not limited to the starting form used for trituration unless if such form is essential for obtaining another form.

In one embodiment, the solvents employed in the preparation of the crystalline forms of the present invention are pharmaceutically acceptable or pharmaceutically non-acceptable solvents, the former being preferred. Pharmaceutically non-acceptable solvents will have to be removed prior to using the crystal form into a pharmaceutical formulation.

The conditions concerning crystallization may be modified in order to improve the crystallization process or to induce precipitation, and without affecting the form of the polymorph obtained. These conditions include bringing the solution, dispersion, or slurry of the compound of formula (I) and the solvent(s) to a desired concentration, cooling it following a defined cooling/temperature curve, adding crystal seeds, bringing the said solution, dispersion, or slurry to a desired temperature, effecting any suitable pressure, removing and/or separating any undesired material or impurities, drying the formed crystals to obtain the polymorphs in a solid state, if such state is desired.

A preferred way of inducing precipitation is to reduce the solubility of the compound of formula (I). The solubility of the compound may be reduced, for example, by cooling the solution. The solubility of the compound of formula (I) may be reduced by adding an anti-solvent.

Bringing the solution, dispersion, or slurry of the compound of formula (I) and solvents to a desired concentration does not necessarily imply an increase in the concentration of the compound of formula (I). In certain cases, a decrease or no change in concentration of the compound of formula (I) could be preferable. The techniques used for obtaining a desired concentration include, for instance, evaporation by atmospheric distillation, vacuum distillation, fractioned distillation, azeotropic distillation, film evaporation, heating, cooling, other techniques well known in the art and combinations thereof. An optional process for obtaining a desired concentration could as well involve the saturation of the solution of the compound of formula (I) and solvent, for example, by adding a sufficient volume of an anti-solvent to the solution to reach the saturation point. Other suitable techniques for saturating the solution include, by way of example, the introduction of additional compound of formula (I) to the solution and/or evaporation of a portion of the solvent from the solution. As referred to herein, a saturated solution encompasses solutions at their saturation points or exceeding their saturation points, i.e. supersaturated. A nearly saturated solution refers to solutions that are near saturation but have not reached their saturation points.

A way to improve the crystallization process of the present invention, in particular of accelerating crystallization, is by seeding with a crystal of the product or scratching the inner surface of the crystallization vessel with a glass rod. Other times, crystallization may occur spontaneously without any inducement. The present invention encompasses both embodiments where crystallization of a particular form of the compound of formula (I) occurs spontaneously, or is induced or accelerated, unless if such inducement or acceleration is critical for obtaining a particular form.

The term “seeding” refers to the addition of a crystalline material to facilitate crystallization. The term “crystal seeds” means powder of a previously obtained crystalline form the compound of formula (I).

By bringing the said solution, dispersion, or slurry to a desired temperature, one will understand the acts of heating, cooling or leaving at ambient temperature. Warming of the solution, dispersion, or slurry may be necessary to completely dissolve the compound of formula (I). Temperatures below 70 degrees Celsius are preferred.

Removing and/or separating any undesired material or impurities may be performed by purification, filtering, washing, precipitation or similar techniques. Separation, for example, can be conducted by known solid-liquid separation techniques. The filtrations can be performed, amongst other methods, by passing the solution, dispersion, or slurry through paper, sintered glass filter or other membrane material, by centrifugation, or using Buchner style filter, Rosenmund filter or plates, or frame press. Preferably, in-line filtration or safety filtration may be advantageously intercalated in the processes disclosed above, in order to increase the purity of the resulting crystal form.

Additionally, filtering agents such as silica gel, Celite®, Arbocel®, dicalite diatomite, or the like, may also be employed to separate impurities from the crystals of interest.

Crystals obtained may be also dried, and such drying process may optionally be used in the different crystallization passages, if more than one crystallization passage is applied. Drying procedures include all techniques known to those skilled in the art, such as heating, applying vacuum, circulating air or gas, adding a desiccant, freeze-drying, spray-drying, evaporating, or the like, or any combination thereof.

Processes for crystallization of polymorphs of the compound of formula (I) may embrace multiple combinations of techniques and variations thereof. Crystallization of the compound of formula (I) may be executed by dissolving, dispersing, or slurrying compound of formula (I) at a suitable temperature in the solvent whereby portion of the said solvent evaporates increasing the concentration of the compound of formula (I) in the said solution, dispersion, or slurry, cooling the said mixture, and optionally washing and/or filtering and drying the resulting crystals of the compound of formula (I). Optionally, crystals of the compound of formula (I) may be prepared by dissolving, dispersing, or slurrying the compound of formula (I) in a solvent medium, cooling the thus obtained solution, dispersion, or slurry and subsequently filtering and drying the obtained polymorph. Another example of preparation of crystal forms of the compound of formula (I) could be by saturating the compound of formula (I) in the solvent medium, and optionally filtering, washing and drying obtained crystals.

Crystal formation may as well involve more than one crystallization process. In certain cases, one, two or more extra crystallization steps may be advantageously performed for different reasons, such as, to increase the quality of the resulting crystal form.

By dissolving, dispersing, or slurrying the compound of formula (I) in the solvent, one may obtain different degrees of dispersion, such as suspensions, slurries or mixtures; or preferably obtain homogeneous one-phase solutions. The term “suspension” refers to a two-phase system consisting of a finely divided solid, i.e. compound of formula (I) in amorphous, crystalline form, or mixtures thereof, dispersed (suspended) in a liquid or dispersing medium, usually the solvent. The term “slurry” refers to a suspension formed when a quantity of powder is mixed into a liquid in which the solid is only slightly soluble (or not soluble). “Slurrying” refers to the making of a slurry.

Optionally, the solvent medium may contain additives, for example dispersing agents, surfactants or other additives, or mixtures thereof of the type normally used in the preparation of crystalline suspensions. The additives may be advantageously used in modifying the shape of crystal by increasing the leniency and decreasing the surface area.

The solvent medium containing the solid may optionally be stirred for a certain period of time, or vigorously agitated using, for example, a high shear mixer or homogenizer or a combination of these, to generate the desired particle size for the organic compound.

Control of precipitation temperature and seeding may be additionally used to improve the reproducibility of the crystallization process, the particle size distribution and form of the product. As such, the crystallization can be effected without seeding with crystals of the compound of the formula (I) or preferably in the presence of crystals of the compound of the formula (I), which are introduced into the solution by seeding. Seeding can also be effected several times at various temperatures. The amount of the seed material depends on the scale of the experiment and can readily be determined by a person skilled in the art. Typically, the amount of seeding material is about 0.1 to 5 weight % of the amount of crystalline material expected from the reaction.

The time for crystallization in each crystallization step will depend on the conditions applied, the techniques employed and/or solvents used.

Breaking up the large particles or aggregates of particles after crystal conversion may additionally be performed in order to obtain a desired and homogeneous particle size. Accordingly, the crystals, powder aggregates and coarse powder of the crystal forms of the compound of formula (I) may be optionally milled and sorted by size after undergoing conversion. Milling or grinding refers to physically breaking up the large particles or aggregates of particles using methods and apparatus well known in the art for particle size reduction of powders. Resulting particle sizes may range from millimeters to nanometers, yielding i.e. nanocrystals, microcrystals. A preferred apparatus for milling or grinding is a fluid energy mill, or micronizer, because of its ability to produce particles of small size in a narrow size distribution.

Pharmaceutical Use of the Crystalline Forms

The present invention further provides a crystalline form of the compound of formula (I), or a mixture of a crystalline form of the compound of formula (I) and the amorphous form of the compound of formula (I), for use as a medicament. In one embodiment, the crystalline form, alone or in any of the above mixtures, for use as a medicament, is selected from Form A.

The present invention further provides the use of crystalline form of the compound of formula (I), or a mixture of a crystalline form of the compound of formula (I) and the amorphous form of the compound of formula (I), in the manufacture of a medicament for the treatment of HCV-related conditions. In one embodiment, the crystalline form, alone or in any of the above mixtures, used in the manufacture of a medicament is selected from Form A.

The present invention provides as well a method of treating a mammal suffering from HCV-related conditions comprising administering a crystalline form of the compound of formula (I), or a mixture of a crystalline form of the compound of formula (I) and the amorphous form of the compound of formula (I), to the mammal in need thereof. In one embodiment, the method of treatment comprises administering a crystalline form, alone or in any of the above mixtures, selected from Form A.

HCV-related conditions include those pathologic conditions brought on by HCV and other pathogenic flaviviruses such as Yellow fever, Dengue fever (types 1-4), St. Louis encephalitis, Japanese encephalitis, Murray valley encephalitis, West Nile virus and Kunjin virus. The diseases associated with HCV include progressive liver fibrosis, inflammation and necrosis leading to cirrhosis, end-stage liver disease, and hepatocellular carcinoma (HCC); and for the other pathogenic flaviviruses the diseases include yellow fever, dengue fever, hemorrhagic fever and encephalitis. HCV and the other pathogenic flaviviruses include both wild-type and mutant strains of HCV.

The term “treatment” refers to any treatment of a pathologic condition in a mammal, particularly a human, and includes one or more of the following acts:
(i) preventing the pathologic condition from occurring in a subject which may be predisposed to the condition but has not yet been diagnosed with the condition and, accordingly, the treatment constitutes prophylactic treatment for the disease condition;
(ii) inhibiting the pathologic condition, i.e., arresting its development;
(iii) relieving the pathologic condition, i.e., causing regression of the pathologic condition; or
(iv) relieving the symptoms mediated by the pathologic condition.

The present invention provides furthermore a pharmaceutical composition comprising a crystalline form of the compound of formula (I), or a mixture of a crystalline form of the compound of formula (I) and the amorphous form of the compound of formula (I), and a pharmaceutically acceptable excipient. In one embodiment, the pharmaceutical composition comprises a crystalline form, alone or in any of the above mixtures, selected from Form A.

Pharmaceutical compositions may be prepared as medicaments to be administered orally, parenterally (including subcutaneously, intramuscularly, and intravenously), rectally, transdermally, bucally, or nasally. Suitable forms for oral administration include powders, granulates, aggregates, tablets, compressed or coated pills, dragees, sachets, hard or gelatin capsules, syrups and suspensions. Suitable forms of parenteral administration include an aqueous or non-aqueous solution or emulsion, while for rectal administration suitable forms for administration include suppositories with hydrophilic or hydrophobic vehicle. For topical administration the invention provides suitable transdermal delivery systems known in the art, and for nasal delivery there are provided suitable aerosol delivery systems known in the art. Although the most suitable administration in any given case will depend on the nature and severity of the condition being treated, the most preferred route of the present invention is oral.

The dosages may be conveniently presented in unit dosage form and prepared by any of the methods well-known in the art. Alternatively, the dosage forms may be presented as one, two, three or four or more subdoses administered at appropriate intervals throughout the day. The unit dosage used is preferably from about 1 mg to about 1000 mg of the compound of formula (I) base equivalent, or from about 5 to about 800 mg, or from about 5 to about 600 mg, or from about 100 to about 600 mg, or from about 200 to about 500 mg.

Pharmaceutical compositions of the present invention comprise the above disclosed crystal forms of the compound of formula (I). The pharmaceutical composition may comprise only a single form of the compound of formula (I), or a mixture of various forms of the compound of formula (I), with or without amorphous form. In addition to the active ingredient(s), the pharmaceutical composition comprises one or more excipients or adjuvants.

Examples of suitable excipients are gum arabic, magnesia, magnesium carbonate, potassium phosphate, lactose, glucose, or starch, in particular, corn starch. Suitable oily excipients or solvents are vegetable or animal oils, such as sunflower oil or cod liver oil. Suitable solvents for aqueous or alcoholic solutions are water, ethanol, sugar solutions, or mixtures thereof. Polyethylene glycols and polypropylene glycols are also useful as further auxiliaries for other administration forms.

In addition to the ingredients particularly mentioned above, the pharmaceutical compositions of the present invention may include other agents conventional in the art having regard to the type of formulation in question, for example those suitable for oral administration may include flavouring agents or taste masking agents.

As used herein, the term “about” has its conventional meaning. In particular embodiments when in relation to a numerical value, it may be interpreted to mean the numerical value ±10%, or ±5%, or ±2%, or ±1%, or ±0.5%, or ±0.1%. In other embodiments, the precise value is meant, i.e. by leaving out the word “about”.

### Examples

The following examples are intended to illustrate the present invention and not to limit it thereto.

Preparation of the Compound of Formula (I)

The compound of formula (I) was prepared as follows.

Synthesis of Compound (I)

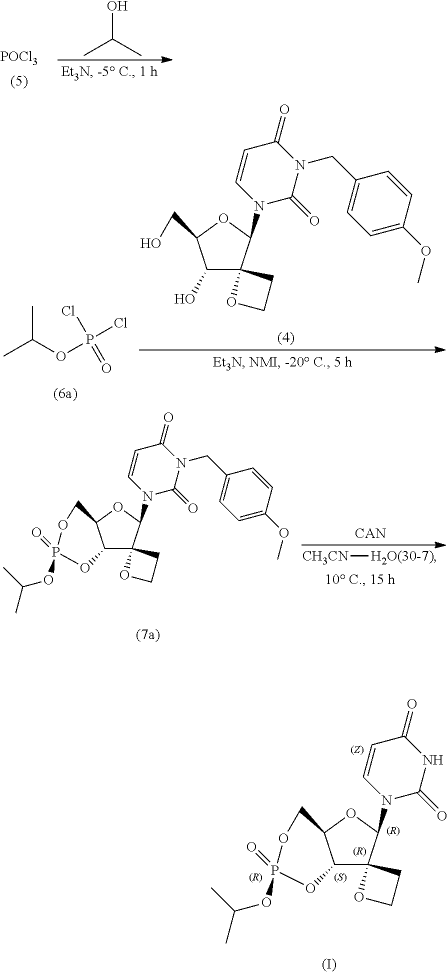

Synthesis of Compound (6a)

A solution of isopropyl alcohol (3.86 mL, 0.05 mol) and triethylamine (6.983 mL, 0.05 mol) in dichloromethane (50 mL) was added to a stirred solution of POCl₃ (5) (5.0 mL, 0.0551 mol) in DCM (50 mL) dropwise over a period of 25 min at −5° C. After the mixture stirred for 1 h, the solvent was evaporated, and the residue was suspended in ether (100 mL). The triethylamine hydrochloride salt was filtered and washed with ether (20 mL). The filtrate was concentrated, and the residue was distilled to give the (6) as a colorless liquid (6.1 g, 69% yield).

Synthesis of Compound (4):

CAS 1255860-33-3 is dissolved in pyridine and 1,3-dichloro-1,1,3,3-tetraisopropyldisiloxane is added. The reaction is stirred at room temperature until complete. The solvent is removed and the product redissolved in CH₂Cl₂ and washed with saturated NaHCO₃ solution. Drying on MgSO₄ and removal of the solvent gives compound (2). Compound (3) is prepared by reacting compound (2) with p-methoxybenzylchloride in the presence of DBU as the base in CH₃CN. Compound (4) is prepared by cleavage of the bis-silyl protecting group in compound (3) using TBAF as the fluoride source.

Synthesis of Compound (7a)

To a stirred suspension of (4) (2.0 g, 5.13 mmol) in dichloromethane (50 mL) was added triethylamine (2.07 g, 20.46 mmol) at room temperature. The reaction mixture was cooled to −20° C., and then (6a) (1.2 g, 6.78 mmol) was added dropwise over a period of 10 min. The mixture was stirred at this temperature for 15 min and then NMI was added (0.84 g, 10.23 mmol), dropwise over a period of 15 min. The mixture was stirred at −15° C. for 1 h and then slowly warmed to room temperature in 20 h. The solvent was evaporated, the mixture was concentrated and purified by column chromatography using petroleum ether/EtOAc (10:1 to 5:1 as a gradient) to give (7a) as white solid (0.8 g, 32% yield).

Synthesis of Compound (I)

To a solution of (7a) in CH₃CN (30 mL) and H₂O (7 mL) was add CAN portion wise below 20° C. The mixture was stirred at 15-20° C. for 5 h under N₂. Na₂SO₃ (370 mL) was added dropwise into the reaction mixture below 15° C., and then Na₂CO₃ (370 mL) was added. The mixture was filtered and the filtrate was extracted with CH₂Cl₂ (100 mL*3). The organic layer was dried and concentrated to give the residue. The residue was purified by column chromatography to give the target compound (8a) as white solid. (Yield: 55%)

¹H NMR (400 MHz, CHLOROFORM-d) δ ppm 1.45 (dd, J=7.53, 6.27 Hz, 6H), 2.65-2.84 (m, 2H), 3.98 (td, J=10.29, 4.77 Hz, 1H), 4.27 (t, J=9.66 Hz, 1H), 4.43 (ddd, J=8.91, 5.77, 5.65 Hz, 1H), 4.49-4.61 (m, 1H), 4.65 (td, J=7.78, 5.77 Hz, 1H), 4.73 (d, J=7.78 Hz, 1H), 4.87 (dq, J=12.74, 6.30 Hz, 1H), 5.55 (br. s., 1H), 5.82 (d, J=8.03 Hz, 1H), 7.20 (d, J=8.03 Hz, 1H), 8.78 (br. s., 1H); ³¹P NMR (CHLOROFORM-d) δ ppm −7.13; LC-MS: 375 (M+1)+

### Example 2: Preparation and Characterization of Form a

The present invention relates to an HCV inhibitor, which is the compound of formula (I) in crystalline form.

The invention in particular concerns the crystalline form of the compound of formula (I) that is denominated as Form A, or in short “Form A”. Two different batches of Form A were prepared. The method used and the resulting purities are disclosed below in Table 3.

TABLE 3
Method and purity of batch 1 and 2 of Form A

Purity

| | | Before | After |
|---|---|---|---|
| | | crystal- | crystal- |
| | Procedure | lization | lization |
| Batch 1 | Dissolve the amorphous | 99.1% | 99.75% |
| (25 g) | compound (I) in 10 V DCM, | | |
| | switch DCM to EA, crystals | | |
| | precipitated out during this | | |
| | operation | | |
| Batch 2 | Add 7 V EA into 1 g amorphous | 97.37% | 99.45% |
| (1 g) | compound (I), crystals came out | | |
| | after stirring 2-3 hrs at r.t | | |

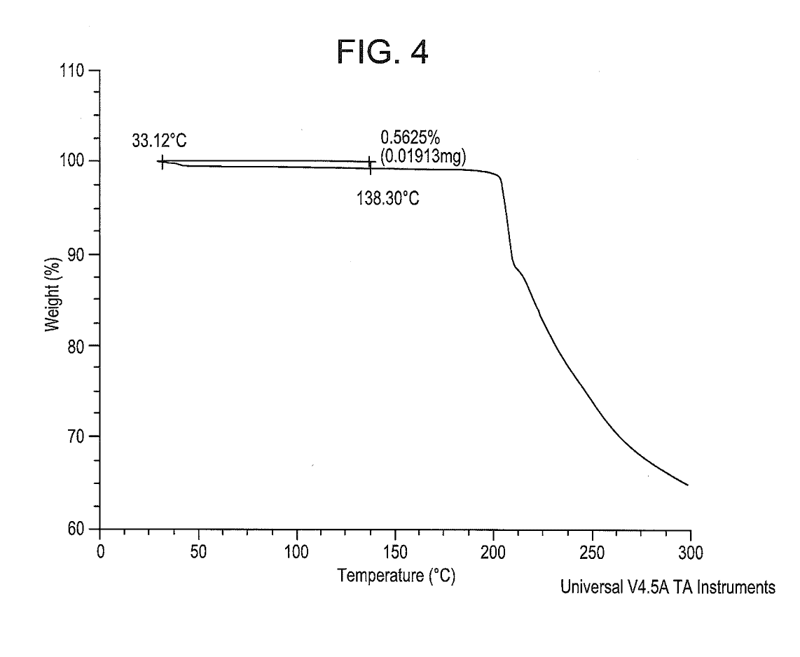

Representative X-ray powder Diffraction (XPRD) pattern representation obtained of Form A is disclosed in FIG. 1. Representative DSC endothermic melt positions or ranges of the crystal Form A are disclosed in FIG. 3. FIG. 4 discloses the thermal gravimetric analysis (TGA) of Form A. FIG. 5 discloses a IR spectrum of Form A.

Powder X-ray Diffraction patterns were obtained on a Rigaku Miniflex Diffraction System (Rigaku MSC Inc.). The powder samples were deposited on a zero-background polished sample holder. A normal focus copper x-ray tube at 0.45 kW equipped with a Ni K filter scanning at 5 degrees/minute from 3.00 to 36.00 degree 2-theta was used as the x-ray source. The data processing was done using Jade 6.0 software. The relative intensities of the XRD peaks can vary depending on the sample preparation technique, crystal size distribution, various filters used, the sample mounting procedure, and the particular instrument employed. Also some new peaks may be seen or some peaks may disappear depending on the type of the machine and settings (including filters). It is commonly accepted that XTD peaks may shift to either side with 2theta −/+0.3 degrees.

DSC data were collected using a Q2000 DSC (TA instruments). Typically 3 mg of sample was used in a semi-hermetically sealed aluminum pan (no pin-hole). The sample was heated from 40° C. to 300° C. at a ramp rate of 10° C./min. The location of DSC peak may be slightly shifted depending on the particle size distribution, type of machine, and the heating rate. The presence of impurities may also change the peak location.

TGA data (FIG. 3) was collected using a Q5000 thermo gravimetric analyzer (TA instruments). 10 mg of sample was deposited on a clean platinum pan and heated from 25° C. to 300° C. at 10° C./min under a 25 ml/min N₂ flow. The location of TGA peaks may be shifted depending on the particle size distribution, type of machine, type and flow rate of purge gas, and the heating rate. The presence of impurities may also change the peak location. Data indicate that Form A is stable from at least 38 to about 140 degrees Celsius.

The IR data were obtained using infrared spectrometry with a Nicolet FT-IR spectrophotometer. The scan parameters for Forms A were as follows:

| | |
|---|---|
| number of scans: | 32 |
| resolution: | 4 cm⁻¹ |
| wavelength range: | 4000 to 400 cm⁻¹ |
| baseline correction: | yes |

### Example 3: Preparation of Form a Using Seed Crystals

A third and fourth batch of Form A were prepared by the following process. 50 mg of amorphous compound of formula (I) starting material from different batches (3 and 4) was added with little seeds of Form A to 10 Volumes of DCM. Then anti-solvent was added with seeds of Form A, to obtain a cloudy appearance. Subsequently, anti-solvent was added until crystallization was complete. The process was carried out a RT

Crystal formation of Form A was observed for different anti-solvents. Best results were obtained with Ethyl acetate, n-Heptane and Isopropyl Acetate. From test results, Ethyl acetate proved to be a preferred anti-solvent.

1. A compound of formula (I): 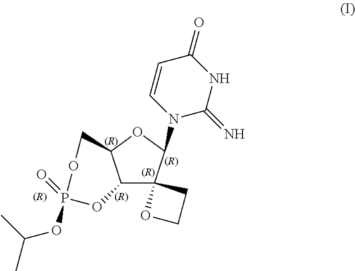
in solid crystalline form.

2. The compound of claim 1 wherein said solid crystalline form is in Form A that has an X-ray powder diffraction pattern comprising peaks at two-theta values of 11.4°±0.3°, 13.1°±0.3°, 14.8°±0.3°, 15.9°±0.3°, 16.5°±0.3°, 18.8°±0.3°, 19.8°±0.3°, 20.7°±0.3° and 21.6°±0.3°.

3. A process for preparing said solid crystalline form as claimed in claim 1, comprising:
a) Dissolving compound of formula (I) in a solvent; and
b) Subsequently adding an anti-solvent until crystal formation is observed.

4. A process for preparing said solid crystalline form as claimed in claim 3 wherein said solvent is selected from the group consisting of Dichloromethane, Tetrahydrofuran, Ethanol and a mixture thereof, and said anti-solvent is selected from the group consisting of Ethyl acetate, n-Heptane, Isopropyl Acetate, Methyl tert-butyl ether and a mixture thereof.

5. A process for preparing said solid crystalline form as claimed in claim 3 wherein said compound of formula (I) is dissolved in least 5 volumes Dichloromethane, and wherein at least 20 volumes of Ethyl acetate, n-Heptane or Ethyl acetate/n-Heptane mixture are subsequently added.

6. A process for preparing said solid crystalline form as claimed in claim 1 comprising:
a) preparing a suspension or slurry of the amorphous form of said compound of formula (I) in Ethyl acetate, n-Heptane or an Ethyl acetate/n-Heptane mixture; and
b) stirring said suspension or slurry for at least one hour.

7. A process as claimed in claim 6, wherein said suspension or slurry is seeded with crystal seeds of said Form A.

8. A process as claimed in claim 3, wherein said preparing and stirring are carried out at room temperature.

9. A pharmaceutical composition comprising a compound as claimed in claim 1.

10. The pharmaceutical composition according to claim 9, wherein said compound is in Form A.

11. A method of treating an HCV infection or conditions associated with an HCV infection, comprising administering a therapeutically effective amount of at least the compound as claimed in claim 1.

12. A process for preparing said solid crystalline form as claimed in claim 2, comprising:
a) Dissolving compound of formula (I) in a solvent; and
b) Subsequently adding an anti-solvent until crystal formation is observed.

13. A process for preparing said solid crystalline form as claimed in claim 3 wherein said solvent is selected from the group consisting of Dichloromethane, Tetrahydrofuran, Ethanol and a mixture thereof, and said anti-solvent is selected from the group consisting of Ethyl acetate, n-Heptane, Isopropyl Acetate, Methyl tert-butyl ether and a mixture thereof.

14. A process for preparing said solid crystalline form as claimed in claim 2 comprising:
a) preparing a suspension or slurry of the amorphous form of said compound of formula (I) in Ethyl acetate, n-Heptane or an Ethyl acetate/n-Heptane mixture; and
b) stirring said suspension or slurry for at least one hour.

15. A pharmaceutical composition comprising a compound as claimed in claim 2.

16. A method of treating an HCV infection or conditions associated with an HCV infection, comprising administering a therapeutically effective amount of at least the compound as claimed in claim 2.
